(19) United States
(12) Patent Application Publication
(10) Pub. No.: US 2020/0239856 A1
(43) Pub. Date: Jul. 30, 2020

(54) CARTILAGE TISSUE PRODUCING METHOD AND CARTILAGE TISSUE

(71) Applicant: GUNZE LIMITED, Ayabe-shi, Kyoto (JP)

(72) Inventors: William J. LANDIS, East Falmouth, MA (US); Robin CHILDS, Canton, OH (US); Narihiko HIRANO, Osaka (JP); Tomokazu FUKUDA, Osaka (JP)

(21) Appl. No.: 16/756,067

(22) PCT Filed: Nov. 29, 2018
(86) PCT No.: PCT/JP2018/044013

§ 371 Date: Apr. 14, 2020

(30) Foreign Application Priority Data
Dec. 4, 2017 (JP) 2017-232716

(51) Int. Cl. C12N 5/071; C12N 5/077; A61L 27/36; A61L 27/18

(52) U.S. Cl. CPC C12N 5/0697; C12N 5/0655; A61L 27/3612; A61L 2430/06; C12N 2501/734; C12N 2533/40; A61L 27/18

(57) ABSTRACT

The present invention aims to provide a method for producing a cartilage tissue, which enables production of a cartilage tissue having an appropriate thickness, form, and mechanical strength, and a cartilage tissue produced by the method for producing a cartilage tissue. Provided is a method for producing a cartilage tissue including a step of seeding a collagenase-treated cartilage tissue piece in the form of a block 50 to 1,000 μm on a side onto a porous substrate composed of a bioabsorbable material.

### TECHNICAL FIELD

The present invention relates to a method for producing a cartilage tissue, which enables production of a cartilage tissue having an appropriate thickness, form, and mechanical strength, and to a cartilage tissue produced by the method for producing a cartilage tissue.

### BACKGROUND ART

Recent progress in cell engineering has enabled culturing of various animal cells including human cells. Research on the reconstruction of human tissues or organs using such cells, that is, so-called regenerative medicine, is progressing rapidly. The point of regenerative medicine is whether cells can proliferate and differentiate into a three-dimensional, living tissue-like structure, and various methods are provided such as a method in which cells and growth factors are used and a method in which a support serving as a scaffold for tissue or organ regeneration is transplanted into a patient. As an example of such a support, a transplantation substrate composed of collagen monofilaments is disclosed in Patent Literature 1.

Also, Patent Literatures 2 and 3 disclose a foam composed of a bioabsorbable material, a substrate for culturing a cardiovascular tissue reinforced with a similar material, and a tubular substrate for nerve regeneration.

Further, Patent Literature 4 discloses a medical material having a skeleton composed of a molded product of a sponge or nonwoven polymer material, wherein a gel having cells dispersed therein is placed inside the skeleton.

One of the subjects of regenerative medicine is regeneration of cartilage tissues. Cartilage tissues are large and thick, and auricular cartilages and the like have complex forms. In addition, cartilage tissues are required to have relatively high mechanical strength. Nevertheless, it has been difficult to produce such large and thick cartilage tissues by methods for producing cartilage tissues employing conventional supports.

### CITATION LIST

### Patent Literature

Patent Literature 1: JP 2003-193328 A
Patent Literature 2: JP 2001-78750 A
Patent Literature 3: JP 2003-19196 A
Patent Literature 4: JP 2003-204807 A

### SUMMARY OF INVENTION

### Technical Problem

In view of the state of the art, the present invention aims to provide a method for producing a cartilage tissue, which enables production of a cartilage tissue having an appropriate thickness, form, and mechanical strength, and also to provide a cartilage tissue produced by the method for producing a cartilage tissue.

### Solution to Problem

The present invention relates to a method for producing a cartilage tissue including a step of seeding a collagenase-treated cartilage tissue piece in the form of a block 50 to 1,000 μm on a side onto a porous substrate composed of a bioabsorbable material.

The present invention is described in detail below.

In conventional methods for producing a cartilage tissue, cartilage cells are isolated from cartilage tissues harvested from living bodies, and the cartilage cells are seeded on substrates. The present inventors studied why production of large thick cartilage tissues is difficult by such conventional methods for producing a cartilage tissue, and found out that damage (physical damage caused by stirring or the like and chemical damage caused by enzyme treatment or the like) accumulates in cartilage cells due to the operation of isolating cartilage cells from cartilage tissues and the damage lowers the proliferating ability. The present inventors made further intensive studies and adopted, instead of seeding cartilage cells isolated from cartilage tissues, cutting harvested cartilage tissues into a block of a size in a certain range, and collagenase-treating the block to prepare a cartilage tissue piece, and seeding the cartilage tissue piece on a porous substrate composed of a bioabsorbable material. Such a process enables extremely highly efficient production of a cartilage tissue having an appropriate thickness, form, and mechanical strength.

The method for producing a cartilage tissue of the present invention includes a step of seeding a collagenase-treated cartilage tissue piece in the form of a block 50 to 1,000 μm on a side onto a porous substrate composed of a bioabsorbable material.

In the case of seeding cartilage cells recovered from raw-material cartilage tissues, damage may accumulate in the cartilage cells during recovery, possibly making regeneration of cartilage tissues difficult. In contrast, accumulation of damage can be minimized and stable regeneration of cartilage tissues is allowed by seeding cartilage tissue pieces prepared by collagenase-treating cartilage tissue pieces cut out in the form of a block 50 to 1,000 μm on a side.

According to conventional common technical knowledge, it had been assumed that cartilage tissues could hardly be produced by seeding cartilage tissue pieces cut out in such a small block. However, the present inventors found that cartilage tissues could be stably produced by using collagenase-treated cartilage tissue pieces.

It should be noted that raw-material cartilage tissues can be harvested by a conventionally known method such as removing the skin, connective tissues, and perichondrium from the auricle obtained from humans and animals. Also, raw-material cartilage tissues may be cartilage tissues with or without perichondrium.

The cartilage tissue pieces are in the form of a block 50 to 1,000 μm on a side. Cartilage tissues can be securely formed in a short time by preparing cartilage tissue pieces of a size in the above specified range. The lower limit of the length of a side of the cartilage tissue pieces is preferably 100 μm and the upper limit thereof is preferably 800 μm. The upper limit is more preferably 400 μm.

Any method may be employed for cutting out the cartilage tissues in the form of a block. Preferred is a method of using a fine processing device for cutting. It is difficult to cut cartilage tissues in such a minute size as 50 to 1,000 μm by manual cutting with a scalpel or the like, and variations occur in the size and form of the cartilage tissue pieces obtained. Moreover, damage may accumulate in cartilage cells due to impact applied upon cutting. The use of cartilage tissue pieces prepared by cutting with a fine processing device enables stable regeneration of cartilage tissues.

The fine processing device is not limited, and a microslicer shown in FIG. 5 can be used, for example. The use of a microslicer enables cartilage tissues to be sliced freely in the directions of X-axis, Y-axis, and Z-axis, whereby cartilage tissue pieces can be easily cut into blocks 50 to 1,000 μm on a side with minimum damage.

The cartilage tissue pieces are subjected to a collagenase treatment. The use of such collagenase-treated cartilage tissue pieces facilitates migration of cartilage cells from cartilage tissue pieces, enabling secure formation of cartilage tissues in a short time. However, collagenase treatment may cause damage to cartilage cells. The collagenase treatment is performed to the extent that cartilage cells are not damaged and migration of cartilage cells from cartilage tissue pieces is facilitated. Specifically, conditions for the collagenase treatment are considered in accordance with the size (length of a side) of cartilage tissue pieces.

The collagenase treatment may be performed by any method. In an exemplary method, cartilage tissue pieces cut into blocks 50 to 1,000 μm on a side are immersed in a buffer containing collagenase dissolved therein (a collagenase solution). Specifically, cartilage tissue pieces are shaken in a collagenase solution having a concentration of 0.3% for 15 to 60 minutes under the conditions of a temperature of 37° C. and a revolution of 160 rpm. As described above, the collagenase treatment is performed to the extent that the cartilage cells are not damaged and migration of cartilage cells from cartilage tissue pieces is facilitated. The conditions for the collagenase treatment are adjusted so that the treatment is performed for a short time in the case of small cartilage tissue pieces and for a long time in the case of large cartilage tissue pieces.

In the step of seeding cartilage tissue pieces, collagenase-treated cartilage tissue pieces are seeded onto a porous substrate composed of a bioabsorbable material.

The seeding may be performed by any method. In an exemplary method, a suspension is prepared by suspending the collagenase-treated cartilage tissue pieces in an appropriate buffer or culture solution, and the suspension is poured onto the porous substrate composed of a bioabsorbable material.

The collagenase-treated cartilage tissue pieces may be seeded at any seeding density. Preferably, the cartilage tissue pieces are seeded in such a manner that the area of cartilage tissue pieces is approximately ¼ of the area of the cartilage tissues to be obtained. Cartilage tissues can be securely formed in a short time when the cartilage tissue pieces are seeded at a density within the above specified range.

The porous substrate onto which the cartilage tissue pieces have been seeded is preferably left to stand still for about 10 minutes until sufficient attachment of the collagenase-treated cartilage tissue pieces is achieved. Also, the collagenase-treated cartilage tissue pieces may be cultured for several hours to several days as needed. A culture solution used in the culturing may be, for example, a serum-added medium obtained by adding approximately 1 to 10% by weight of fetal bovine serum to a common culture solution such as MEM or DMEM.

The porous substrate composed of a bioabsorbable material may have any form. For example, it may be in the form of a nonwoven fabric or sponge. In particular, a nonwoven fabric is preferred as it has excellent handleability and serves as a scaffold in the process where, after seeding of cartilage tissue pieces, cartilage cells migrate out of the seeded cartilage tissue pieces and proliferate to form cartilage tissues.

The bioabsorbable material constituting the porous substrate is not limited, and examples thereof include polyglycolide, polylactides (D-, L-, and DL-isomer), polycaprolactone, glycolic acid-lactic acid (D-, L-, and DL-isomer) copolymers, glycolic acid-s-caprolactone copolymers, lactic acid (D-, L-, and DL-isomer)-ε-caprolactone copolymers, and poly(p-dioxanone). These may be used alone or in combination of two or more.

Among them, a nonwoven fabric composed of polyglycolide has an excellent property of allowing penetration of cells, thereby exerting excellent effects on cartilage tissue regeneration. In the case where a nonwoven fabric composed of polyglycolide is immersed in saline of 37° C., approximately 14 days are required until its tensile strength is reduced to half of that before the immersion. Owing to such degradability, the nonwoven fabric is gradually degraded and absorbed during the period of cell proliferation and tissue regeneration to allow construction of regenerated tissues even inside the nonwoven fabric, resulting in construction of good-quality cartilage tissues.

Polyglycolide as used herein refers to glycolide polymers such as polyglycolic acid. Copolymers of polyglycolide and other bioabsorbable components such as lactic acid, ε-caprolactone, p-dioxanone, and trimethylene carbonate may also be used as long as the effect of the present invention is not impaired. Provided that the effect of the present invention is not impaired, polyglycolide may be used in the form of a mixture with other bioabsorbable materials such as polylactide.

In the case where polyglycolide is used as a bioabsorbable material constituting the nonwoven fabric, the weight average molecular weight of the polyglycolide is not limited. The lower limit thereof is preferably 30,000 and the upper limit thereof is preferably 250,000. When the weight average molecular weight of the polyglycolide is less than 30,000, the mechanical strength may be insufficient, while when the weight average molecular weight exceeds 250,000, degradation speed in the living body may be lowered, possibly causing foreign-body reactions. The lower limit of the weight average molecular weight of the polyglycolide is more preferably 50,000 and the upper limit thereof is more preferably 220,000.

The lower limit of the average pore size of the nonwoven fabric is preferably 20 μm and the upper limit thereof is preferably 50 μm. The nonwoven fabric having such an average pore size enables easy production of especially large and thick cartilage tissues. The reason for this is probably because cells can easily penetrate into pores having a size of 20 to 50 μm of the nonwoven fabric and form tissues by proliferation and differentiation in the layer into which they have penetrated. In contrast, when the average pore size is smaller than 20 μm, efficient penetration of cells may not be allowed, whereas when the average pore size is larger than 50 μm, cells can penetrate but sufficient proliferation and differentiation of the cells attached to the nonwoven fabric may not be allowed because the cells are too distant from each other. The lower limit of the average pore size of the nonwoven fabric is more preferably 22 μm, still more preferably 24 μm and the upper limit thereof is more preferably 40 μm, still more preferably 30 μm.

As used herein, the average pore size of a nonwoven fabric refers to an average pore size as measured by the bubble point method.

Measurement of the pore size distribution of a nonwoven fabric by the bubble point method is described.

In the bubble point method, a liquid that well soaks into a membrane to be measured is allowed to be absorbed into the pores of the membrane in advance, and the membrane is set in an instrument such as one shown in FIG. 1. Then, air pressure is applied from the underside of the membrane and the minimum pressure (bubble point) at which generation of air bubbles is observed on the membrane surface is measured. The pore size distribution is estimated based on the relational formula of the surface tension of the liquid and the bubble point (FIG. 2).

Specifically, a nonwoven fabric to be measured is allowed to absorb a wetting liquid (e.g., fluorine-based solvent, product name: Porofil™), and then set in an instrument such as one shown in FIG. 1 (e.g., Porometer 3G available from BEL Japan, Inc.) in such a manner that the nonwoven fabric as a test piece is in a circular shape with a diameter of 25 mm. Air pressure is applied from the underside of the nonwoven fabric and the minimum pressure (bubble point) at which generation of air bubbles is observed on the membrane surface is measured.

In the formula for calculating the pore size shown in FIG. 2, γ represents the surface tension of the wetting liquid, θ represents the contact angle of the wetting liquid on the nonwoven fabric material, and ΔP represents the bubble point pressure.

The nonwoven fabric may have any average fiber diameter. The lower limit of the average fiber diameter is preferably 10 μm and the upper limit thereof is preferably 50 μm. With the average fiber diameter of the nonwoven fabric within this range, the average pore size can be easily adjusted within the above specified range. The lower limit of the average fiber diameter of the nonwoven fabric is more preferably 15 μm and the upper limit thereof is more preferably 40 μm.

The average fiber diameter of the nonwoven fabric can be obtained as follows: Based on an image of the nonwoven fabric taken at 1000× magnification by a scanning electron microscope, the diameter of the mid-point of all fibers with measurable diameters is measured, followed by calculating the average of the measured values.

Fibers constituting the nonwoven fabric may be either monofilaments or multifilaments; however, in order to securely hold tissue pieces while maintaining flexibility, a nonwoven fabric composed of multifilaments is more preferred.

In the case where the fibers constituting the nonwoven fabric are multifilaments, the fineness of single fiber constituting the multifilaments is not limited. In view of the tissue piece retainability and flexibility of the nonwoven fabric, the lower limit is preferably 1 denier and the upper limit is preferably 10 denier.

In the case where the fibers constituting the nonwoven fabric are multifilaments, the number of single fibers constituting a multifilament is not limited. In view of the tissue piece retainability and flexibility of the nonwoven fabric, the lower limit is preferably 8 and the upper limit is preferably 15.

The nonwoven fabric may be produced by any method, and any conventionally known method such as electrospinning deposition, melt blowing, needle punching, spunbonding, flash spinning, hydroentanglement, air-laid methods, thermal bonding, resin bonding, or wet methods may be employed. Among them, needle punching is suitable.

A nonwoven fabric obtained by the above method may be used after being subjected to thermal compression to have an adjusted thickness or average pore size. In view of the property of allowing penetration of cells, however, a nonwoven fabric not subjected to thermal compression is preferably used. A nonwoven fabric not subjected to thermal compression has a downy appearance and exhibit an excellent property of allowing penetration of cells.

The lower limit of the thickness of the porous substrate is preferably 150 μm and the upper limit thereof is preferably 1,000 μm. With the thickness of smaller than 150 μm, only fragile tissues may be regenerated. With the thickness exceeding 1,000 μm, nutrient diffusion inside the porous substrate may be insufficient, leading to necrosis of the tissues. The lower limit of the thickness of the porous substrate is more preferably 250 μm and the upper limit thereof is more preferably 800 μm.

The porous substrate may also be used together with a porous substrate having an average pore size of 5 to 20 μm (hereinbelow, also referred to as a “small-pore-size porous substrate”). The small-pore-size porous substrate allows smooth passage of body fluids, blood, and the like, while hardly allowing passage of cells. Thus, use of the porous substrate in combination with a small-pore-size porous substrate can prevent cartilage tissue pieces and cells from falling off the porous substrate, while supplying the cartilage tissue pieces seeded onto the porous substrate and the cells migrating from the cartilage tissue pieces with sufficient nutrients, thereby enabling more efficient production of cartilage tissues. The lower limit of the average pore size of the small-pore-size porous substrate is preferably 6 μm, more preferably 7 μm and the upper limit thereof is preferably 18 μm, more preferably 16 μm.

The small-pore-size porous substrate may have any form, and it may be in the form of a nonwoven fabric or sponge.

A bioabsorbable material constituting the small-pore-size porous substrate may be a similar bioabsorbable material to that used for the porous substrate. The bioabsorbable material constituting the small-pore-size porous substrate may be the same as or different from the bioabsorbable material constituting the porous substrate.

The porous substrate is preferably fixed to a mold, composed of a bioabsorbable material (hereafter, also simply referred to as a “mold”) so as to be combined and integrated with the mold. The mold plays a role of shaping the form of cartilage tissues to be obtained, while imparting sufficient mechanical strength to the cartilage tissues. Seeding of cartilage tissue pieces onto a porous substrate combined and integrated with a mold enables production of thicker cartilage tissues having a higher mechanical strength.

Any bioabsorbable material may be used to constitute the mold, and a similar bioabsorbable material to that used for the porous material may be used.

In consideration of the role played by the mold, i.e., shaping the form of cartilage tissues to be obtained and imparting sufficient mechanical strength to the cartilage tissues, a bioabsorbable material that requires a longer time for degradation than the bioabsorbable material constituting the porous substrate is preferably chosen as the bioabsorbable material constituting the mold. With use of a mold constituted by a bioabsorbable material that requires a longer time for degradation than the bioabsorbable material constituting the porous substrate, the form of cartilage tissues can be maintained during proliferation of cartilage cells and regeneration of cartilage tissues along with gradual degradation and absorption of the porous substrate.

For example, in the case where polyglycolide is used as a bioabsorbable material constituting the porous substrate, polycaprolactone is preferable as a bioabsorbable material constituting the mold. Polycaprolactone has moderate mechanical strength and flexibility in addition to requiring a longer time for degradation than polyglycolide.

In the case where polycaprolactone is used as a bioabsorbable material constituting the mold, polycaprolactone may interfere with the formation of cartilage tissues on or around the mold due to its high hydrophobicity. In such a case, the mold composed of polycaprolactone is preferably subjected to hydrophilization treatment in advance.

The hydrophilization treatment is not limited, and conventionally known hydrophilization treatment may be employed. In particular, treatment with alcohol such as ethanol is preferred as it is simple and has less influence on living bodies.

Examples of the form of the mold include a film, a lattice, a mesh, and a concentric circle.

When a cartilage tissue having a specific form such as auricular cartilage is produced, the mold is molded to conform to the form of a cartilage tissue to be produced, whereby a cartilage tissue in any form can be obtained.

The porous substrate is preferably combined and integrated with the mold. If the mold and the porous substrate are not combined and integrated, the porous substrate may be detached partly or entirely from the mold during the process of seeding cartilage tissue pieces onto the porous substrate or transplantation into tissues or organs. When the porous substrate is detached even partly from the mold, a cell pool may be formed in a void formed in the area of detachment, possibly preventing normal regeneration of tissues or organs.

Combination and integration of the mold and the porous substrate herein means that the mold and the porous substrate are not detached from each other even they are folded upon transplantation of the cartilage tissues obtained into tissues or organs.

Examples of a method for combining and integrating the mold and the porous substrate include a method in which a part of the surface of the mold or porous substrate is melted with heat to attach them together, a method in which the mold and the porous substrate are attached together with a medical adhesive, and a method in which a part of the surface of the mold or porous substrate is melted with a solvent to attach them together.

The mold may be arranged in such a manner as to surround the porous substrate or to be wrapped in the porous substrate. Preferably, the mold is arranged in such a manner as to be wrapped in the porous substrate. With such arrangement, cartilage tissues having a more appropriate thickness and form and higher mechanical strength can be produced.

FIG. 3 shows schematic views illustrating exemplary embodiments of a porous substrate combined and integrated with the mold.

In a porous substrate 1 integrated and combined with a mold shown in FIG. 3(a), a mold 12 is arranged to surround a rectangular porous substrate 11. In a porous substrate 1′ integrated and combined with a mold shown in FIG. 3(b), two rectangular porous substrates 11 are arranged to sandwich a mold 12 therebetween.

In a porous substrate 2 integrated and combined with a mold shown in FIG. 4(a), a mold 22 is shaped in the form of an external ear, and a porous substrate 21 is placed inside the mold 22. In a porous substrate 2′ integrated and combined with a mold shown in FIG. 4(b), two porous substrates 21 are arranged to sandwich a mold 22 shaped in the form of an external ear therebetween.

Transplantation of cartilage tissues produced by the method for producing a cartilage tissue of the present invention enables regeneration of cartilage tissues having an appropriate thickness, form, and mechanical strength.

The present invention also encompasses a cartilage tissue including a porous substrate composed of a bioabsorbable material and a collagenase-treated cartilage tissue piece in the form of a block 50 to 1,000 μm on a side seeded on the porous substrate.

### Advantageous Effects of Invention

The present invention provides a method for producing a cartilage tissue which enables production of a cartilage tissue having an appropriate thickness, form, and mechanical strength, and a cartilage tissue produced by the method for producing a cartilage tissue.

### BRIEF DESCRIPTION OF DRAWINGS

FIG. 1 shows a schematic view explaining how to measure the pore size distribution of a porous substrate by the bubble point method.

FIG. 2 shows a schematic view explaining how to estimate the pore size distribution of a porous substrate based on data obtained by the bubble point method.

FIG. 3 shows schematic views illustrating exemplary embodiments of a porous substrate combined and integrated with a mold used in the method for producing a cartilage tissue of the present invention.

FIG. 4 shows schematic views illustrating exemplary embodiments of a porous substrate combined and integrated with a mold used in the method for producing a cartilage tissue of the present invention.

FIG. 5 shows a schematic view illustrating an exemplary microslicer that can be used as a fine processing device.

FIG. 6 shows a safranin-staining image (a) and a Verhoeff's staining image (b) of a cross-section of a nonwoven fabric onto which cartilage tissue pieces have been seeded, taken 10 weeks after transplantation as in Example 1.

FIG. 7 shows safranin-staining images (a) and Verhoeff's staining images (b) of cross-sections of nonwoven fabrics onto which respective collagenase-treated cartilage tissue pieces have been seeded, taken 10 weeks after transplantation as in Example 2.

FIG. 8 shows a safranin-staining image (a) and a Verhoeff's staining image (b) of a cross-section of a nonwoven fabric onto which untreated cartilage tissue pieces have been seeded, taken 10 weeks after transplantation as in Comparative Example 1.

FIG. 9 shows a safranin-staining image (a) and a Verhoeff's staining image (b) of a cross-section of a nonwoven fabric onto which cartilage tissue pieces have been seeded, taken 10 weeks after transplantation as in Example 3.

FIG. 10 shows safranin-staining images (a) and Verhoeff's staining images (b) of cross-sections of nonwoven fabrics onto which respective collagenase-treated cartilage tissue pieces have been seeded, taken 10 weeks after transplantation as in Example 4.

FIG. 11 shows a safranin-staining image (a) and a Verhoeff's staining image (b) of a cross-section of a nonwoven fabric onto which untreated cartilage tissue pieces have been seeded, taken 10 weeks after transplantation as in Comparative Example 2.

FIG. 12 shows a safranin-staining image (a) and a Verhoeff's staining image (b) of a cross-section of a nonwoven fabric onto which cartilage tissue pieces in the form of a block 100 μm on a side subjected to collagenase treatment for 15 minutes have been seeded, taken 20 weeks after transplantation as in Example 5.

FIG. 13 shows a safranin-staining image (a) and a Verhoeff's staining image (b) of a cross-section of a nonwoven fabric onto which cartilage tissue pieces in the form of a block 200 μm on a side subjected to collagenase treatment for 15 minutes have been seeded, taken 20 weeks after transplantation as in Example 5.

FIG. 14 shows a safranin-staining image (a) and a Verhoeff's staining image (b) of a cross-section of a nonwoven fabric onto which cartilage tissue pieces in the form of a block 400 μm on a side subjected to collagenase treatment for 60 minutes have been seeded, taken 20 weeks after transplantation as in Example 5.

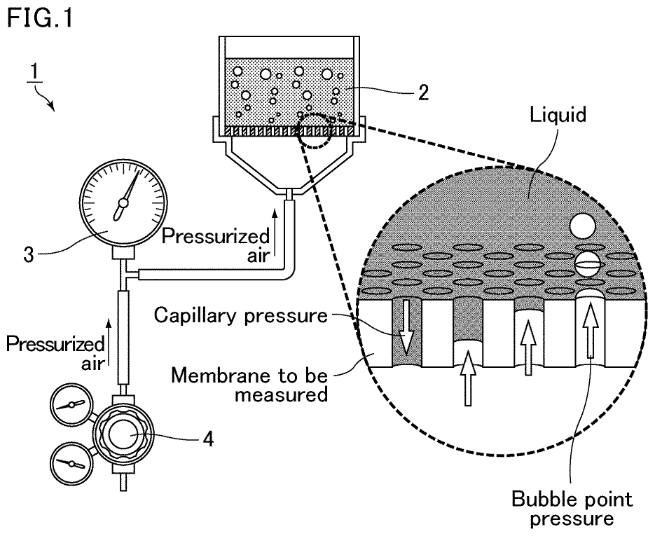

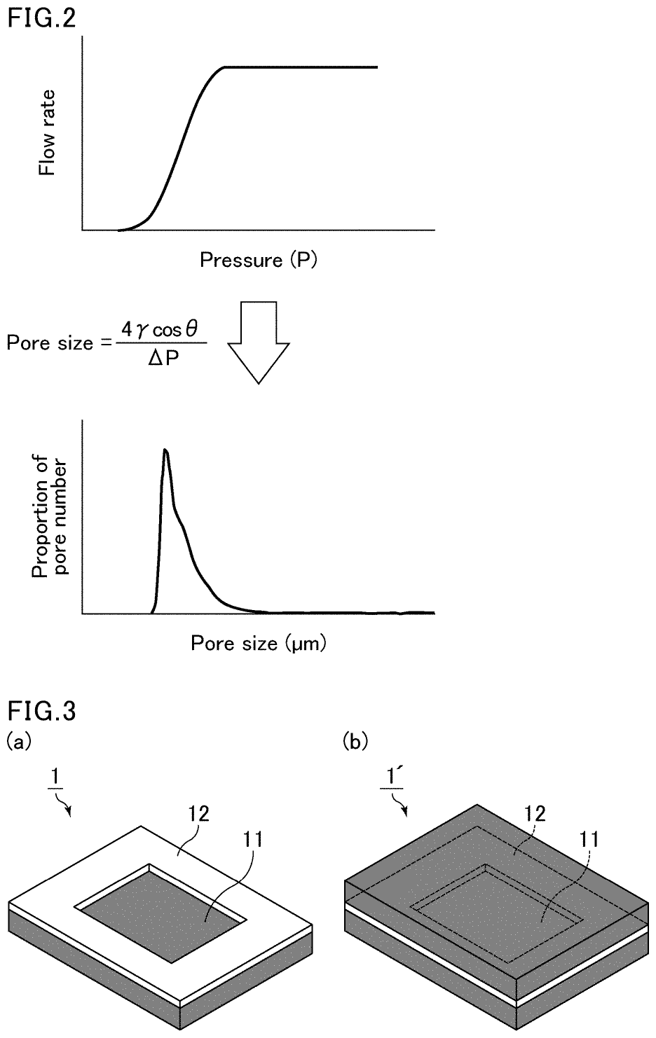

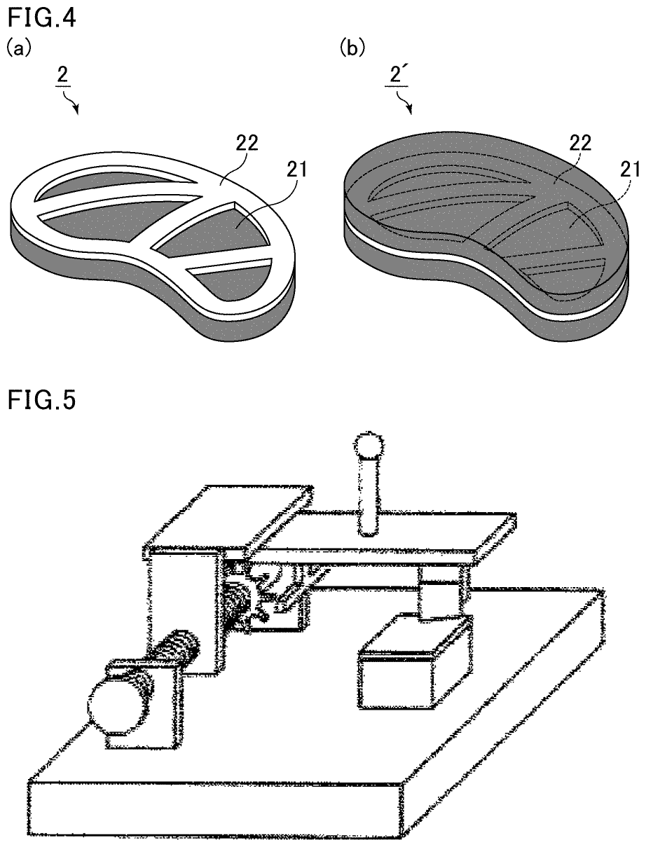

### DESCRIPTION OF EMBODIMENTS

Embodiments of the present invention are described in more detail with reference to, but not limited to, Examples.

### EXAMPLE 1

### (1) Preparation of Nonwoven Fabric

A downy nonwoven fabric having an average fiber diameter of approximately 16 μm and a thickness of approximately 0.5 mm was obtained by the following method: Polyglycolide having a weight average molecular weight of 250,000 was used as a bioabsorbable material and spun into filaments, and then made into a nonwoven fabric by needle punching.

The obtained layer into which cells penetrate was allowed to absorb a wetting liquid of a fluorine solvent (product name: Profil™), and then set in Prometer 3G, the product of BEL Japan, Inc., in such a manner that the layer as a test piece was in a circular shape with a diameter of 25 mm. Air pressure was applied from the underside of the layer into which cells penetrate and the minimum pressure (bubble point) at which generation of air bubbles was observed on the membrane surface was measured. Based on the bubble point thus obtained, a graph showing the pore size distribution of the nonwoven fabric was obtained. The average pore size was 28 μm as calculated from the graph.

### (2) Preparation and Seeding of Cartilage Tissue Pieces

The skin, connective tissues, and perichondrium were removed from human auricular cartilage obtained by excision for the treatment of prominent ear to give a human auricular cartilage having a length of 5 mm, a width of 5 mm, and a thickness of 1 mm. The human auricular cartilage was sliced with a fine processing device into cartilage tissue pieces approximately 800 μm on a side.

The obtained cartilage tissue pieces were subjected to a collagenase treatment, which included shaking in a collagenase solution having a concentration of 0.3% for 60 minutes under the conditions of a temperature of 37° C. and a revolution of 160 rpm.

The collagenase-treated cartilage tissue pieces were suspended in 50 μL phosphate buffer and evenly seeded onto the nonwoven fabric using a pipette. The cartilage tissue pieces were seeded in such a manner that the area of the cartilage tissue pieces seeded was 5 mm² with respect to 1 cm² of the area of the cartilage tissues to be obtained.

### (3) Transplantation and Evaluation of Cartilage Tissue Regeneration

The nonwoven fabric onto which the cartilage tissue pieces thus obtained had been seeded was transplanted into 4 to 6-week-old athymic mice (an average body weight of 28 g, male). After general anesthesia, an incision was made in the posterior region of neck and the nonwoven fabric were transplanted under the skin. Ten weeks after the transplantation, the mice were sacrificed and respective samples were collected.

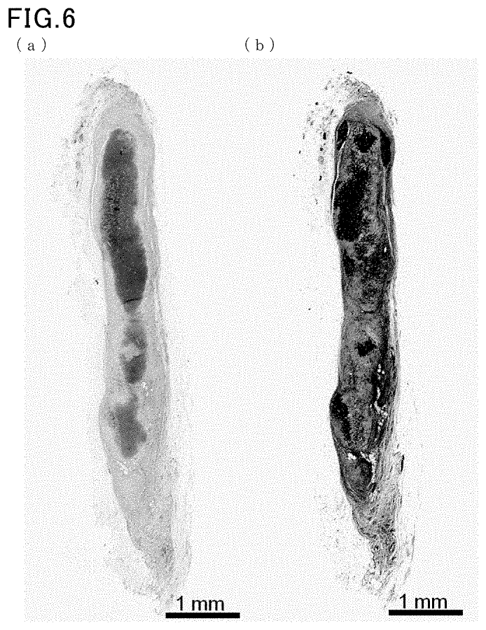

FIG. 6 shows a safranin-staining image (a) and Verhoeff's staining image (b) of cross-sections of the nonwoven fabrics onto which cartilage tissue pieces have been seeded, taken 10 weeks after transplantation. According to FIG. 6(a), it was conformed that proteoglycans was produced and regenerated cartilages were induced. In FIG. 6(b), the area stained black indicates transplanted cartilage pieces and the area stained slightly dark indicates regenarated cartilage. Induction of regenerated cartilages was confirmed also from the observation that the area stained black occupied only a small area. Further, polyglycolide constituting the nonwoven fabrics was completely degraded.

### EXAMPLE 2

Seeding of the cartilage tissue pieces onto nonwoven fabrics, transplantation, and evaluation of cartilage tissue regeneration were performed in the same manner as in Example 1, except that the conditions for collagenase treatment performed on cartilage tissue pieces were set to (1) 15 minutes, (2) 30 minutes, (3) 45 minutes, and (4) 60 minutes.

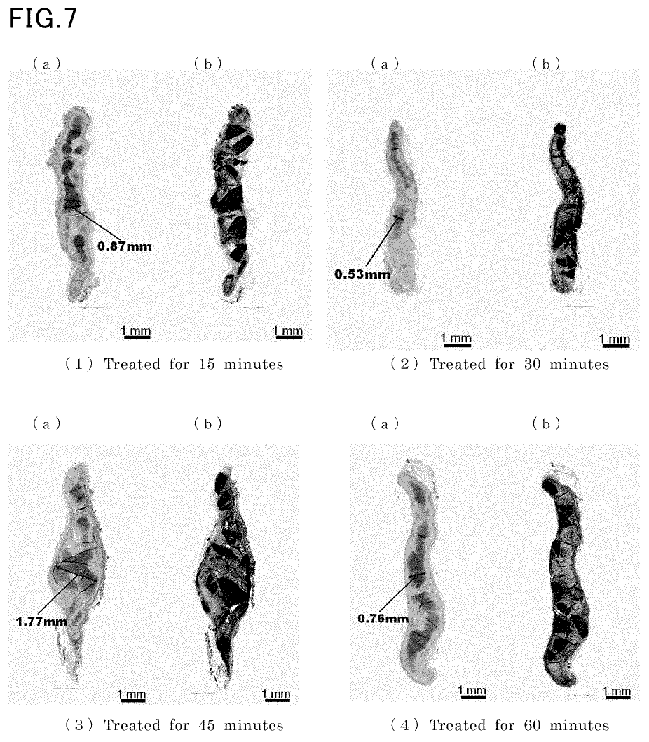

FIG. 7 shows a safranin-staining image (a) and a Verhoeff's staining image (b) of cross-sections of the nonwoven fabrics onto which respective collagenase-treated cartilage tissue pieces have been seeded, taken 10 weeks after transplantation.

### COMPARATIVE EXAMPLE 1

Seeding of the cartilage tissue pieces onto nonwoven fabrics, transplantation, and evaluation of cartilage tissue regeneration were performed in the same manner as in Example 1, except that cartilage tissue pieces were not subjected to collagenase treatment.

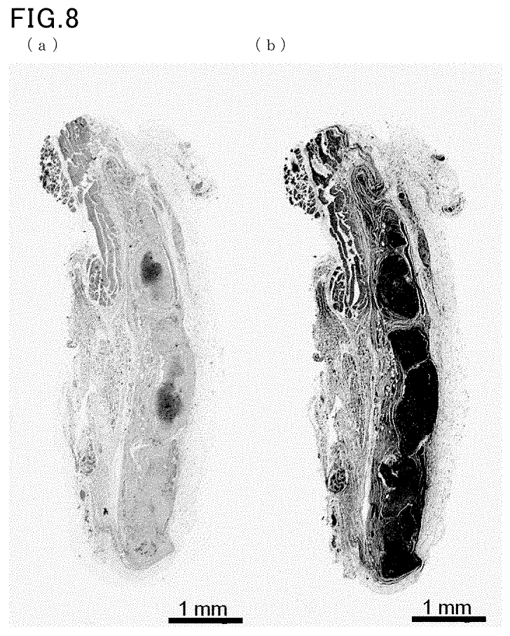

FIG. 8 shows a safranin-staining image (a) and a Verhoeff's staining image (b) of cross-sections of nonwoven fabrics onto which cartilage tissue pieces have been seeded, taken 10 weeks after transplantation. According to FIG. 8, it was confirmed that production of proteoglycans was poor and regenerated cartilage was hardly induced in Comparative Example 1. Though residual cartilage tissue pieces were observed, an absorption image was observed.

### EXAMPLE 3

### (1) Preparation of Nonwoven Fabric

A downy nonwoven fabric having an average fiber diameter of approximately 16 μm and a thickness of approximately 0.5 mm was obtained by the following method: Polyglycolide having a weight average molecular weight of 250,000 was used as a bioabsorbable material and spun into filaments, and then made into a nonwoven fabric by needle punching.

The obtained layer into which cells penetrate was allowed to absorb a wetting liquid of a fluorine solvent (product name: Profil™), and then set in Porometer 3G, the product of BEL Japan, Inc., in such a manner that the layer as a test piece was in a circular shape with a diameter of 25 mm. Air pressure was applied from the underside of the layer into which cells penetrate and the minimum pressure (bubble point) at which generation of air bubbles was observed on the membrane surface was measured. Based on the bubble point thus obtained, a graph showing the pore size distribution of the nonwoven fabric was obtained. The average pore size was 28 μm as calculated from the graph.

### (2) Preparation of Small-Pore-Size Nonwoven Fabric

A small-pore-size nonwoven fabric having an average fiber diameter of approximately 2 μm and a thickness of approximately 50 μm was obtained by the following method: Polyglycolide having a weight average molecular weight of 250,000 was used as a bioabsorbable material, and made into a nonwoven fabric by melt blowing.

The average pore size of the obtained small-pore-size nonwoven fabric was calculated by the bubble point method, and was 12 μm.

### (3) Lamination of Nonwoven Fabric and Small-Pore-Size Nonwoven Fabric

The small-pore-size nonwoven fabric having a thickness of 50 μm and the nonwoven fabric having a thickness of 0.5 mm were stacked on each other and stitched at the four corners with a 5-0 nylon thread. Thus, a laminate was obtained.

### (4) Preparation and Seeding of Cartilage Tissue Pieces

The skin, connective tissues, and perichondrium were removed from human auricular cartilage obtained by excision for the treatment of prominent ear to give a human auricular cartilage having a length of 5 mm, a width of 5 mm, and a thickness of 1 mm. The human auricular cartilage was sliced with a fine processing device into cartilage tissue pieces approximately 800 μm on a side.

The cartilage tissue pieces thus obtained were subjected to a collagenase treatment, which included shaking in a collagenase solution having a concentration of 0.3% for 60 minutes under the conditions of a temperature of 37° C. and a revolution of 160 rpm.

The collagenase-treated cartilage tissue pieces were suspended in 50 μL phosphate buffer and evenly seeded onto the nonwoven fabric side of the laminate using a pipette. The cartilage tissue pieces were seeded in such a manner that the area of the cartilage tissue pieces to be seeded is 5 mm² with respect to 1 cm² of the area of the cartilage tissues to be obtained.

### (5) Transplantation and Evaluation of Cartilage Tissue Regeneration

The nonwoven fabric onto which the cartilage tissue pieces thus obtained had been seeded was transplanted into 4 to 6-week-old athymic mice (an average body weight of 28 g, male). After general anesthesia, an incision was made in the posterior region of neck and the cartilage tissues were transplanted under the skin. Ten weeks after transplantation, the mice were sacrificed and respective samples were collected.

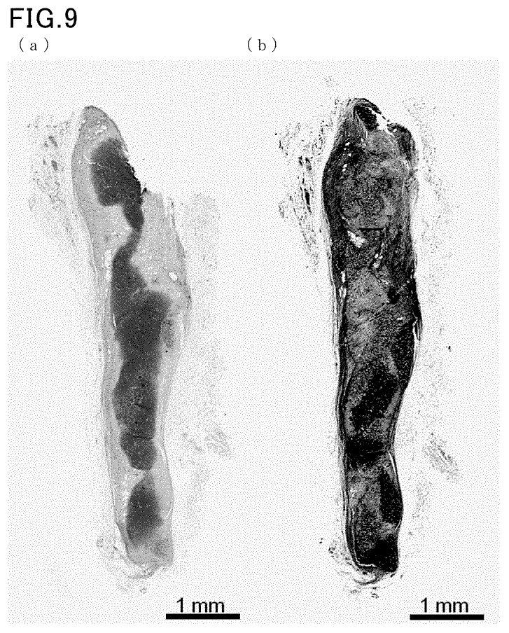

FIG. 9 shows a safranin-staining image (a) and a Verhoeff's staining image (b) of a cross-section of a nonwoven fabric onto which cartilage tissue pieces have been seeded, taken 10 weeks after transplantation. According to FIG. 9(a), it was confirmed that a large amount of proteoglycan was produced and regenerated cartilages were more induced than Example 1. Induction of regenerated cartilages was confirmed also from the observation that the area stained black occupied only a small area in FIG. 9(b). Further, polyglycolide constituting the nonwoven fabric was completely degraded.

### EXAMPLE 4

Seeding of the cartilage tissue pieces onto a nonwoven fabric, transplantation, evaluation of cartilage tissue regeneration were performed in the same manner as in Example 3, except that the conditions for collagenase treatment performed on cartilage tissue pieces were set to (1) 15 minutes, (2) 30 minutes, (3) 45 minutes, and (4) 60 minutes.

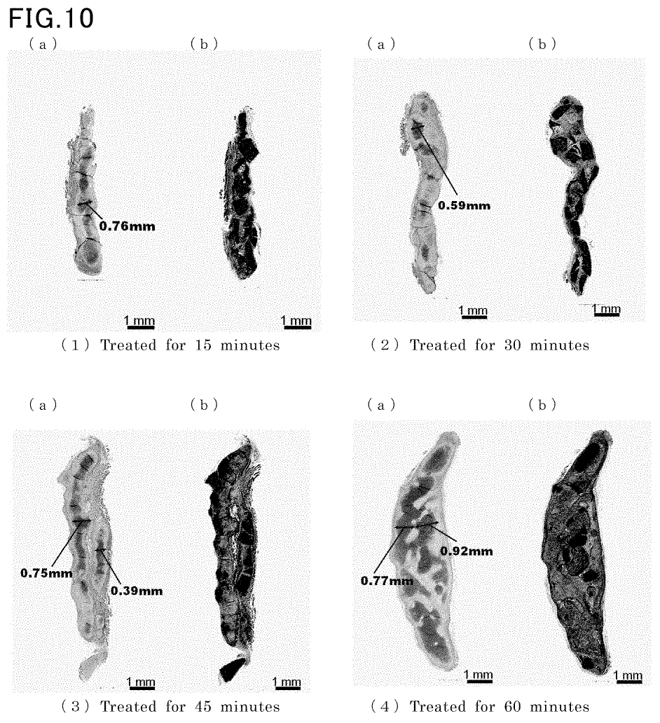

FIG. 10 shows safranin-staining images (a) and Verhoeff's staining images (b) of cross-sections of the nonwoven fabrics onto which respective collagenase-treated cartilage tissue pieces have been seeded, taken 10 weeks after transplantation.

### COMPARATIVE EXAMPLE 2

Seeding of the cartilage tissue pieces onto a nonwoven fabric, transplantation, and evaluation of cartilage tissue regeneration were performed in the same manner as in Example 3, except that cartilage tissue pieces were not subjected to collagenase treatment.

FIG. 11 shows a safranin-staining image (a) and a Verhoeff's staining image (b) of a cross-section of a nonwoven fabric onto which cartilage tissue pieces have been seeded, taken 10 weeks after transplantation. According to FIG. 11, it was confirmed that production of proteoglycan was poor and regenerated cartilages were hardly induced in Comparative Example 2. Though residual cartilage tissue pieces were observed, an absorption image was observed.

### EXAMPLE 5

### (1) Preparation and Seeding of Cartilage Tissue Pieces

The skin, connective tissues, and perichondrium were removed from an ear obtained by excision from a canine to give a canine auricular cartilage. The canine auricular cartilage was sliced with a fine processing device into cartilage tissue pieces in the form of a block 100 μm on a side, a block 200 μm on a side, and a block 400 μm on a side.

The obtained cartilage tissue pieces of respective sizes were subjected to a collagenase treatment, which included shaking in a collagenase solution having a concentration of 0.3% for 0 minutes, 15 minutes, and 60 minutes under the conditions of a temperature of 37° C. and a revolution of 160 rpm.

The collagenase-treated cartilage tissue pieces of respective sizes were each suspended in 50 μL phosphate buffer, thereby preparing suspensions. The suspensions were evenly seeded onto the nonwoven fabric side of laminates prepared by the same method as in Example 3 using a pipette. The cartilage tissue pieces were seeded in such a manner that the area of the cartilage tissue pieces seeded was 5 mm² with respect to 1 cm² of the area of the cartilage tissues to be obtained.

### (2) Transplantation and Evaluation of Cartilage Tissue Regeneration

The nonwoven fabrics onto which the cartilage tissue pieces were seeded were transplanted to the canine from which one ear was excised in the above step (1). After general anesthesia, an incision was made on the head and the cartilage tissues were transplanted under the skin. Twenty weeks after transplantation, the canine was sacrificed and respective samples were collected.

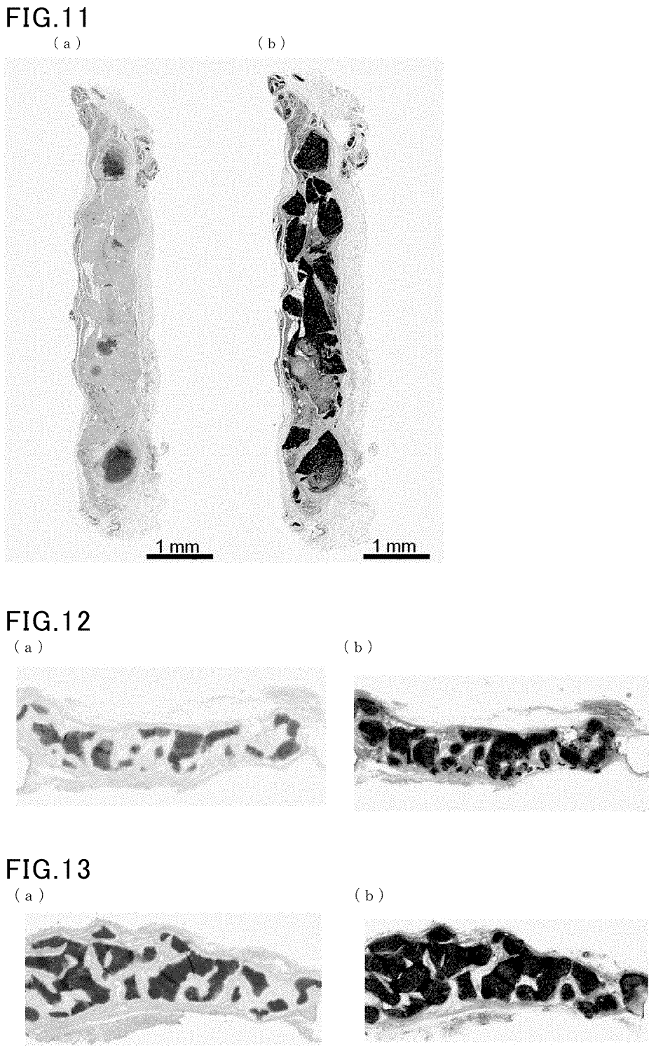

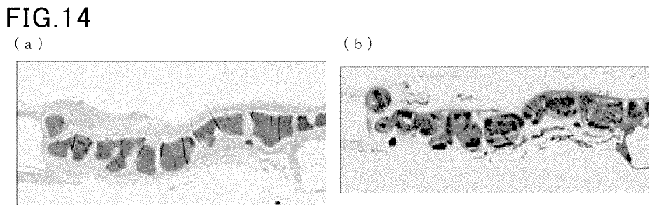

Cross-sectional slices of the nonwoven fabrics 20 weeks after transplantation were prepared, and were subjected to safranin staining and Verhoeff's staining. FIG. 12 shows a safranin-staining image (a) and a Verhoeff's staining image (b) of a cross-section of a nonwoven fabric onto which cartilage tissue pieces in the form of a block 100 μm on a side subjected to collagenase treatment for 15 minutes have been seeded, taken 20 weeks after transplantation. FIG. 13 shows a safranin-staining image (a) and a Verhoeff's staining image (b) of a cross-section of a nonwoven fabric onto which cartilage tissue pieces in the form of a block 200 μm on a side subjected to collagenase treatment for 15 minutes have been seeded, taken 20 weeks after transplantation. FIG. 14 shows a safranin-staining image (a) and a Verhoeff's staining image (b) of a cross-section of a nonwoven fabric onto which cartilage tissue pieces in the form of a block 400 μm on a side subjected to collagenase treatment for 60 minutes have been seeded, taken 20 weeks after transplantation.

In safranin staining, cartilage tissues are stained red. In FIGS. 12 to 14, many cartilage tissue parts stained red can be observed, which confirms regeneration of cartilage tissues. Image processing of the safranin-stained image enables calculation of the area of regenerated cartilage tissue parts (area of the cartilage tissue parts stained red). This method clarified that in the case of cartilage tissue pieces in the form of a block 100 μm on a side and a block 200 μm on a side, collagenase treatment for 15 minutes allowed especially favorable cartilage regeneration. It also clarified that in the case of cartilage tissue pieces in the form of a block 400 μm on a side, collagenase treatment for 60 minutes allowed especially favorable cartilage regeneration.

The results show that the optimum collagenase treatment conditions are different according to the size (length of one side) of the cartilage tissue pieces.

### EXAMPLE 6

### (1) Preparation and Seeding of Cartilage Tissue Pieces

The skin, connective tissues, and perichondrium were removed from an ear obtained by excision from a canine to give a canine auricular cartilage tissue. The obtained canine auricular cartilage was cut into blocks of 1 cm×1 cm (1 cm²), and then further sliced with a fine processing device into cartilage tissue pieces in the form of a block 400 μm on a side.

The obtained cartilage tissue pieces were subjected to a collagenase treatment, which included shaking in a collagenase solution having a concentration of 0.3% for 15 minutes under the conditions of a temperature of 37° C. and a revolution of 160 rpm.

The collagenase-treated cartilage tissue pieces were suspended in 50 μL phosphate buffer, thereby preparing a suspension. The whole suspension was evenly seeded using a pipette onto the nonwoven fabric side of a laminate prepared by the same method as in Example 3 and cut to a size of 2 cm×2 cm (4 cm²).

### (2) Transplantation and Evaluation of Cartilage Tissue Regeneration

The nonwoven fabric onto which the cartilage tissue pieces had been seeded was transplanted to the canine from which one ear was excised in the above step (1). After general anesthesia, an incision was made on the head and the cartilage tissues were transplanted under the skin. Twenty weeks after transplantation, the canine was sacrificed and the nonwoven fabric was collected.

A cross-sectional slice of the nonwoven fabric 20 weeks after transplantation was prepared, and subjected to safranin staining. Formation of cartilage tissue parts stained red was observed on the entire surface of the nonwoven fabric.

This result shows that larger cartilage tissues can be regenerated with only a small amount of cartilage tissue pieces.

### INDUSTRIAL APPLICABILITY

The present invention can provide a method for producing a cartilage tissue which enables production of a cartilage tissue having an appropriate thickness, form, and mechanical strength, and also provide a cartilage tissue produced by the method for producing a cartilage tissue.

### REFERENCE SIGNS LIST

1, 1′ porous substrate combined and integrated with mold
11 rectangular porous substrate
12 mold
2, 2′ porous substrate combined and integrated with mold
21 porous substrate
22 mold shaped in the form of external ear

1. A method for producing a cartilage tissue comprising
a step of seeding a collagenase-treated cartilage tissue piece in the form of a block 50 to 1,000 μm on a side onto a porous substrate composed of a bioabsorbable material.

2. The method for producing a cartilage tissue according to claim 1,
wherein the cartilage tissue piece is in the form of a block 100 to 800 μm on a side.

3. The method for producing a cartilage tissue according to claim 1,
wherein the porous substrate is a nonwoven fabric having an average pore size of 20 to 50 μm.

4. The method for producing a cartilage tissue according to claim 1,
wherein the bioabsorbable material constituting the porous substrate is polyglycolide.

5. The method for producing a cartilage tissue according to claim 1,
wherein the porous substrate is fixed to a mold composed of a bioabsorbable material to combine and integrate with the mold.

6. The method for producing a cartilage tissue according to claim 5,
wherein the bioabsorbable material constituting the mold is polycaprolactone.

7. A cartilage tissue comprising
a porous substrate composed of a bioabsorbable material and
a collagenase-treated cartilage tissue piece in the form of a block 50 to 1,000 μm on a side seeded on the porous substrate.
